(19) United States
(12) Patent Application Publication
(10) Pub. No.: US 2006/0293215 A1
(43) Pub. Date: Dec. 28, 2006

(54) FRAGRANCE MATERIALS AND PROCESSES FOR PRODUCING SUCH MATERIALS

(76) Inventor: John Morgan Sizemore, Dallas, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(21) Appl. No.: 11/160,426

(22) Filed: Jun. 23, 2005

(51) Int. Cl. A61K 8/00

(52) U.S. Cl. 512/3

(57) ABSTRACT

Fragrance materials and processes for producing such fragrance materials including pre-treating an inorganic substrate with electromagnetic radiation to form a pre-treated inorganic substrate and contacting the pre-treated inorganic substrate with a fragrance composition or an ink, or with a mixture or a fragrance composition and an ink.

### FIELD OF THE INVENTION

The present invention relates generally to fragrance materials and processes for producing fragrance materials.

### BACKGROUND OF THE INVENTION

A vide variety of pleasant fragrances are commercially available in today's marketplace for use in the home or office, ranging from natural products such as flowers and eucalyptus leaves, to synthetic materials, such as candles, incense, spices, and various sprays and oils. Sprays and oils containing a fragrance typically, due to their high evaporation rate, are embodied in solid carriers, such as for example plastic, ceramics, woods, gels, and waxes (such as for example candle wax). Other common carries include mixtures of dried plant materials, which is commonly sold and known as “potpourri.”

In addition, salt-based fragrance products to which fragrance oils have been applied (e.g., bath salts or crystals) are commercially available. These products (sometimes referred to as “crystal” potpourri) are generally produced by physically applying a fragrance oil or perfume directly to the surface of the salt product, for example by spraying. However, it is generally known that salt, due to its physical structure, does not absorb fragrances well. As such, these products do not retain their fragrance over long periods of time and require periodic additional applications of fragrance to the salt crystal. In addition, because these products are produced by applying the fragrance oil or perfume directly to the surface of the salt, the products are generally sticky, wet or may contain a residue, which may be undesirable.

In addition, salt-based fragrance products available on the commercial market may contain color. When color is desired, color (such as in the form of a dye and/or mica) is also applied directly to the surface of the salt. However, over time, the colors of these products fade at a much higher rate when exposed to direct sunlight or atmospheric conditions. In addition, these products often “bleed” their color onto the surfaces that they touch, as a result of the topical application of the coloring agent.

As such, development of a fragrance product that is able to maintain its color and fragrance over time by employing environmentally safe and available products, would be a significant contribution to the art. In addition, development of a fragrance product that exhibits improved physical properties would also be a significant contribution in the art. Further, development of processes for producing such a fragrance product would be a significant contribution to the art.

### SUMMARY OF THE INVENTION

Accordingly, it is an object of the present invention to provide a process for producing a fragrance material that is able to maintain its color and fragrance over time by employing environmentally safe and available products. A further object of the present invention is to provide fragrance materials produced by such a process.

In one embodiment of the present invention, a process is provided for producing a fragrance material including the steps of pre-treating an inorganic substrate with electromagnetic radiation to form a pre-treated inorganic substrate and contacting the pre-treated inorganic substrate with a fragrance composition or an ink, or with a mixture of a fragrance composition and an ink. In another embodiment of the present invention, a fragrance material produced by such a process is provided.

In another embodiment of the present invention, a process is provided for producing a fragrance material including the steps of contacting an inorganic substrate with a mixture containing a fragrance composition and an ink, and subjecting the inorganic substrate to electromagnetic radiation sufficient to effect a cure thereof. In another embodiment of the present invention, a fragrance material produced by such a process is provided.

Still another embodiment of the present invention is a fragrance material containing a solid-semi-porous inorganic substrate containing sodium chloride having been exposed to electromagnetic radiation, a fragrance composition dispersed throughout the inorganic substrate, and an ink dispersed throughout the inorganic substrate.

Those skilled in the art will further appreciate the advantages and superior features of the invention mentioned above together with other important aspects upon reading the detailed description which follows in conjunction with the appended claims.

### DETAILED DESCRIPTION OF THE INVENTION

Fragrance materials of the present invention generally may include an inorganic substrate, a fragrance composition, and optionally, a colorant such as an ink. The term, “inorganic substrate,” is used generally to refer to any substance that does not contain compounds of carbon and are capable of being modified by electromagnetic radiation and bonded with a fragrance composition and/or colorant. Inorganic substrates useful in the present invention include hydrated chloride salts of Group IA and 2A elements of the periodic chart of the elements, including but not limited to sodium chloride, potassium chloride, magnesium chloride, and calcium chloride. In one embodiment of the present invention, the inorganic substrate is sodium chloride or common salt. Inorganic substrates useful in the present invention may be derived from natural sources (such as for example, from seawater). Generally, inorganic based substrates useful in the present invention are porous, and exhibits a crystalline structure, which may be modified.

Inorganic substrates useful in the present invention may be any color. Generally, the color of the inorganic substrate ranges from colorless (when in a pure form), to white, gray, or brownish (typical of rock salt (halite)), depending on the purity of the substrate. Inorganic substrates useful in the present invention may be derived from various processes including but not limited to solar evaporation, which involves evaporating sea water and obtaining the salt product. Inorganic substrates useful in the present invention generally may be pure or contain impurities, either natural or synthetic, which may or may not be soluble in water. In addition, inorganic substrates useful in the present invention may contain water, which if present, is generally present in an amount less than 5 weight percent, based on the total weight of the inorganic substrate. Examples of impurities commonly found in inorganic substrates useful in the present invention include, but are not limited to, calcium, magnesium and sulfates. Generally, if present, the impurities are present in an amount of less than 2 percent, but may be more or less than 2 percent depending on the inorganic substrate employed. In one embodiment of the present invention, impurities are present in an amount of less than about 2 percent, based on the total weight of the inorganic substrate. In another embodiment of the present invention, impurities are present in an amount in the range of less than about 1 weight percent, based on the total weight of the inorganic substrate. In another embodiment of the present invention, impurities are present in an amount in the range of from about 0.01 to about 0.5 weight percent, based on the total weight of the inorganic substrate. In another embodiment of the present invention, impurities are present in an amount in the range of from about 0.02 to about 0.3 weight percent, based on the total weight of the inorganic substrate.

In one embodiment of the present invention, the inorganic substrate contains sodium chloride or NaCl, generally in an amount in the range of from about 95 percent by weight, to greater than about 99 percent by weight, based on the total weight of the inorganic substrate. In one embodiment of the present invention, the inorganic substrate contains in the range of from about 99.1 to about 99.9 percent sodium chloride, based on the weight of the inorganic substrate. In another embodiment of the present invention, the inorganic substrate contains in the range of from about 99.3 to about 99.9 percent sodium chloride, based on the weight of the inorganic substrate.

Inorganic substrates useful in the present invention may also be any shape or size. Examples of shapes for substrates of the present invention include, but are not limited to, spherical,. cubical, cylindrical, round, and variations thereof. In one embodiment of the present invention, the substrate is non-uniform in shape and size and contains jagged edges and crevices. The size of the substrate employed may be any size, but generally has an average size in the range of from about 10 mm to about 90 mm in diameter. In one embodiment of the present invention, the inorganic substrate is approximately 1 inch in length , 0.5 inch in depth, and 0.5 inches in height. However, it is understood that inorganic substrates of any size and shape may be employed. Representative samples of inorganic substrates useful in the present invention include salt crystals, harvested from the Atlantic Ocean off the coast of Brazil and commercially available from Saltworks, Inc. (Redmond, Wash.), as well as salt products mined from the earth.

The term “fragrance” or “fragrance composition” as used herein refers to any odoriferous material having a vapor pressure below atmospheric pressure at ambient temperatures. Fragrance compositions useful in the present invention generally are in a liquid form at ambient temperatures. A wide variety of chemicals are known for fragrance uses, including materials such as aldehydes, ketones, and esters. More commonly, naturally occurring plant and animal oils and exudates comprising complex mixtures of various chemical components are known for use as fragrances. Fragrance compositions useful in the present invention may be relatively simple in their composition or can comprise highly sophisticated complex mixtures of natural and synthetic chemical components, all chosen to provide any desired odor. Any material which exudes a pleasant or otherwise desirable odor can be used in the present invention.

Examples of fragrance compositions useful in the present invention include, but are not limited to, animal perfumes such as musk oil, civet, cestrum, ambergris, and the like and combinations thereof, plant perfumes such as sandalwood oil, neroli oil, bergamot oil, lemon oil, lavender oil, sage oil, rosemary oil, peppermint oil, eucalyptus oil, menthol, camphor, verbena oil, citronella oil, salvia oil, clove oil, chamomile oil, sandalwood oil, costus oil, labdanum oil, broom extract, carrot seed extract, jasmine extract, mimosa extract, narcissus extract, olibanum extract, rose extract and the like and combinations thereof, and chemical substances such as acetophenone and naphthalene derivatives, alpha-amylcinnamic aldehyde, anethole, anisaldehyde, benzyl acetate, benzyl alcohol, benzyl propionate, borneol, cinnamyl acetate, cinnamyl alcohol, citral citronnellal, cumin aldehyde, cyclamen aldehyde, decanol, ethyl butyrate, ethyl caprate, ethyl cinnamate, ethyl vanillin, eugenol, geraniol, hexenol, alpha-hexylcinnamic aldehyde, hydroxycitrolnellal, indole, iso-amyl acetate, iso-amyl acetate, iso-amyl iso-valeratek iso-eugenol, linalol, linalyl acetate, p-methylacetophenone, methyl anthranilate, methyl dihydroasmonate, methyl eugenol, methyl-beta-naphthol ketone, methylphenylcarbinyl acetate, musk ketol, musk xylol, 2,56-nanodinol, gamma-nanolactone, phenylacetoaldehydodimethyl acetate, beta.-phenylethyl alcohol, 3,3,5-trimethylcyclohexanol, gamma-undecalactone, undecenal, vanillin and the like, and mixtures thereof. Fragrance compositions useful in the present invention also may provide a visual clue, that is, the fragrance material may include a coloring agent. Typically this will be a dye that is compatible with the fragrance composition.

Fragrance compositions useful in the present invention generally are present in an upon of up to about 8 percent by weight, although greater amounts may be employed. In one embodiment of the present invention, the fragrance composition is present in an amount in the range of from about 0.1 weight percent to about 8 percent by weight, based on the total weight of the inorganic substrate. In another embodiment of the present invention, the fragrance composition is present in an amount in the range of from about 0.2 weight percent to about 7 weight percent, based on the total weight of the inorganic substrate. In another embodiment of the present invention, the fragrance compositions is present in the range of from about 0.3 weight percent to about 6 weight percent, based on the total weight of the inorganic substrate. In another embodiment of the present invention, the fragrance compositions is present in the range of from about 0.4 weight percent to about 5 weight percent, based on the weight of the inorganic substrate.

As used herein, the term “ink” and the phrase “ink composition” are used interchangeably and refer to any pigment-carrying or color-providing composition that may be useful for infusing into an inorganic substrate, such as for example, containing sodium chloride. Ink compositions useful in the present invention may vary in chemical components depending on the desired performance characteristics of the ink employed, including by way of example but not limited to, consistency, tack, viscosity, drying characteristics, gloss, chemical resistance, durability, and color.

Ink compositions useful in the present invention generally are present in an amount of up to about 8 percent by weight, based on the total weight of the inorganic substrate. In one embodiment of the present invention, the ink composition is present in an amount in the range of from about 0.1 to about 8 weight percent, based on the weight of the inorganic substrate. In another embodiment of the present invention, the ink composition is present in an amount in the range of from about 0.2 to about 7 weight percent, based on the weight of the inorganic substrate. In another embodiment of the present invention, the ink composition is present in an amount in the range of from about 0.3 to about 6 weight percent, based on the weight of the inorganic substrate. In another embodiment of the present invention, the ink composition is present in an amount in the range of from about 0.4 to about 5 weight percent, based on the weight of the inorganic substrate.

Examples of suitable ink compositions include those that contain one or more carrying or vehicular components, and one or more colorants. Examples of vehicular components of inks include, but are not limited to water, vegetable oils (e.g., linseed oil), mineral oils, and fatty acids. Generally, the vehicular component of the ink is present in an amount in the range of from about 5 to about 25 weight percent, based on the total weight of the ink. In another embodiment of the present invention, the vehicular component of the ink is present in an amount in the range of from about 7 to about 20 weight percent, based on the total weight of the ink. In another embodiment of the present invention, the vehicular component of the ink is present in an amount in the range of from about 9 to about 18 weight percent, based on the total weight of the ink. In one embodiment of the present invention, the vehicle component of the ink is linseed oil.

Inks useful in the present invention may also contain various pigments. Generally, any chemical that imparts color to the vehicle component of the ink may be used as a pigment. Any of the customary inorganic and organic pigments may be used in the ink compositions of the present invention. Generally, one or more pigments are contained in ink compositions useful in the present invention in an amount generally in the range of from about 1 to about 25 weight percent, based on the weight of the ink. In one embodiment of the present invention, the pigment is present in an amount in the range of from about 2 to about 20 weight percent, based on the total weight of the ink. In another embodiment of the present invention, the pigment is present in an amount in the range of from about 4 to about 18 weight percent, based on the total weight of the ink. In another embodiment of the present invention, the pigment is present in an amount in the range of from about 7 to about 18 weight percent, based on the total weight of the ink.

In one embodiment of the present invention, the ink composition employs a pigment that is a “stock color.” A “stock color” pigment or ink component as used herein is a color which does not have to be specially formulated or manufactured (i.e., one which is generally available on demand). Cyan, magenta, yellow, and black are examples of stock colors. A “custom color” pigment or ink component (also known as a “custom-color ink”) as used herein is any color not a stock color, that may be provided in an ink. Formulations of “custom colors” may be formulated in accordance with published color-mixing guides or guides, including but not limited to color models, CMYK, RGB, LAB, HSB, Hesachome, and Pantone (such as for example, the “PANTONE® Formula Guide” by Pantone, Inc. (Carlstadt, N.J.), as well as other color models. A custom-color ink prepared according to published information may be useful where a particular color of fragrance material is desired. The present invention allows an end user to accurately select a desired color (or desired mixture of colors) by referencing a color-mixing guide. For example, an end user may wish to obtain a fragrance product to match a predetermined color such as a selected PANTONE® color (or mixtures of multiple PANTONE®) colors). Methods of the present invention may be used to produce such a customized ink formulation to be employed in the fragrance material.

Generally, the PANTONE®) MATCHING SYSTEM.RTM color matching system is based upon a total of fourteen different standard colors of pigmented inks. These fourteen different standard color of ink are currently known as (1) PANTONE® .RTM yellow ink, (2) PANTONE®.RTM yellow 012 ink, (3) PANTONE®.RTM orange 021 ink, (4) PANTONE®.RTM warm red ink, (5) PANTONE®.RTM red 32 ink, (6) PANTONE®.RTM rubine red ink, (7) PANTONE®.RTM rhodamine red ink, (8) PANTONE®.RTM purple ink, (9) PANTONE®.RTM violet ink, (10) PANTONE®.RTM blue 072 ink, (11) PANTONE®.RTM reflex blue ink, (12) PANTONE®.RTM process blue ink, (13) PANTONE® green ink, and (14) PANTONE®.RTM black ink. The PANTONE® MATCHING SYSTEM.RTM color matching system is one of the most popular color matching systems in the printing industry, especially in the United States. The color gamut of the PANTONE® MATCHING SYSTEM.RTM color matching system is based upon mixing varying combinations of different colored inks to form a single ink of a particular color that is thereafter employed in fragrance materials of the present invention.

In addition, inks useful in the present invention may contain one or more binders which bind the pigment with the vehicle component. Examples of suitable binders include, but are not limited to, alkyd, phenol-formaldehyde or other synthetic resins (such as for example, rosin modified phenolic resins, and alkyd resins). Generally, binders are present in an amount in the range of from about 5 to about 25 weight percent, based on the total weight of the ink. In one embodiment of the present invention, the binder is present in an amount in the range of from about 7 to about 20 weight percent, based on the total weight of the ink. In another embodiment of the present invention, the binder is present in an amount in the range of from about 8 to about 18 weight percent, based on the total weight of the ink.

In addition, ink compositions useful in the present invention may include other components such as for example, organic solvents, water, rheology modifiers, ultraviolet (UV) components, waxes, and the like and combinations thereof, that may affect body, tack, or drying characteristics. For example, cobalt, manganese and lead-based soaps may be used to catalyze the oxidative drying reaction of the ink components. Some types of inks on the other hand dry by evaporation of a volatile solvent, or by applying a UV source, rather than by oxidation and polymerization of a drying oil or resin. It will be appreciated by the skilled artisan that other additives known in the art may be included in the ink compositions of the present invention, so long as such additives do not significantly detract from the benefits of the present invention. Illustrative examples of ink additives include, without limitation, pour point depressants, surfactants, wetting agents, waxes, emulsifying agents and dispersing agents, defoamers, antioxidants, UV absorbers, dryers (e.g., for formulations containing vegetable oils), flow agents and other rheology modifiers, gloss enhancers, and anti-settling agents. When included, additives are typically included in amounts of at least about 0.001% of the ink composition, and may be included in amounts of about 30 percent by weight or more of the ink composition.

In addition, ink compositions useful in the present invention may also contain one or more reducing agents, such as for example, petroleum derived hydrocarbons. A suitable example of a reducing agent useful in the present invention is a commercially available product offered under the trade designation “52 Oil” (available from Magie Brothers, Inc.). Reducing agents are generally used to modify the viscosity of a printing ink prior to application. In typical printing applications (such as for example, newspaper printing), the amount of reducing agent employed is generally less than 30 percent by weight. Reducing agents may be generally employed in greater amounts in the present invention, generally, in the range of from about 40 to about 70 percent by weight, based on the total weight of the ink. In one embodiment of the present invention, the reducing agent is present in an amount in the range of from about 45 weight percent to about 65 weight percent, based on the total weight of the ink. In another embodiment of the present invention, the reducing agent is present in an amount in the range of from about 50 weight percent to about 60 weight percent, based on the total weight of the ink.

In accordance with the present invention, an inorganic substrate may be first exposed to electromagnetic radiation or energy in the form of electromagnetic waves (also called radiant energy, or light). Various forms of electromagnetic radiation may be employed and are characterized by the wavelength, including cosmic rays, gamma rays from radioactive disintegration of atomic nuclei, x-rays, ultraviolet rays, visible light rays, infrared, microwave and radio and electric rays. In one embodiment of the present invention, ultraviolet light is used as the electromagnetic radiation source, utilizing various lamps including, but not limited to “H”, “D”, “V”, “X”, “M” and “A” lamps, mercury arc lamps, and xenon arc lamps (such as those commercially available from Loctite Corporation, Rocky Hill, Conn., Fusion UV Curing Systems, Buffalo Grove, Ill. or Spectroline, Westbury, N.Y.). In another embodiment of the present invention, microwaves are employed having a frequency in the range of from about 1 GHz to about 300 GHz.

Electromagnetic radiation sources useful in the present invention may employ the use of reflectors and/or filters, so as to focus the emitted radiation onto a specific portion of the inorganic substrate. Similarly, electromagnetic radiation may be generated directly in a steady fashion or in an intermittent fashion so as to minimize the degree of heat build-up in the substrate. In one embodiment of the present invention, the source of electromagnetic radiation may remain stationary while the inorganic substrate passes through its path. In another embodiment of the present invention, the inorganic substrate may remain stationary while the source of electromagnetic radiation passes over or around the inorganic substrate. In another embodiment of the present invention, both the inorganic substrate and the electromagnetic radiation source traverse one another, or both remain stationary.

The energy employed in the present invention may be delivered by exposing the inorganic substrate to a less powerful source of electromagnetic radiation for a longer period of time, through for example multiple passes, or alternatively, by exposing the substrate to a more powerful source of electromagnetic radiation for a shorter period of time. In addition, each of those multiple passes may occur with a source at different energy intensities. In any event, those persons of skill in the art should choose an appropriate source of electromagnetic radiation depending on the particular substrate, and position that source at a suitable distance therefrom which, together with the length of exposure, to optimize transformation. Also, it may be desirable to use a source of electromagnetic radiation that is delivered in an intermittent fashion, such as by pulsing or strobing, without causing excessive heat build-up.

Generally, the amount, duration, and frequency of electromagnetic radiation applied to the inorganic substrate varies, depending on the desired end product. Higher or lower radiation intensities, greater or fewer exposures thereto and length of exposure and/or greater or lesser distances of the source of radiation to the substrate may be required, depending on the desired end product. It should be appreciated that the frequency of the alternating current can be varied, thereby causing the applied electromagnetic radiation to penetrate the substrate at various depths. During the time in which the inorganic substrate is exposed to an electromagnetic radiation source, the substrate will be exposed to amount of energy, typically measured in KJ/m², determined by parameters including: the size, type and geometry of the source, the duration of the exposure to electromagnetic radiation, the intensity of the radiation (and that portion of radiation emitted within the region appropriate to effect curing), the absorbency of electromagnetic radiation by any intervening materials, such as other substrates employed in the process, and the distance the substrate lies from the source of radiation. Persons of ordinary skill in the art should readily appreciate that the process may be optimized by choosing appropriate values for these parameters in view of the particular substrate employed, or the desired results.

The product produced in accordance with the present invention, generally contains high porosity, and is able to be infused with an ink composition, a fragrance composition, or a mixture of an ink composition and a fragrance composition. While not wishing to be bound by theory, the inorganic substrate may be chemically modified upon exposure to electromagnetic radiation, thus allowing the inorganic substrate, preferably modified inorganic substrate, to readily accept and chemically bond to the chemical components contained in the fragrance compositions and/or ink compositions employed.

Upon exposure of the inorganic substrate with electromagnetic radiation, a modified inorganic substrate is produced. Thereafter, the inorganic substrate, preferably modified inorganic substrate may be contacted with a fragrance composition, an ink composition, or a mixture or mixtures of both, as well as (or optionally) other additives, including coloring agents and/or mica, to produce a fragrance material that is able to maintain its color and fragrance over a long period of time. Any method for contacting the inorganic substrate, preferably a modified inorganic substrate, with a fragrance composition, an ink composition or mixture thereof may be employed, including but not limited to, spraying, rolling, dispensing, submerging, and topical coating. Thereafter, the inorganic substrate may be allowed to dry at ambient temperature, or may be subjected to thermal radiation (such as for example, a conventional or kiln-type oven), or further, may be subjected to electromagnetic radiation, such as for example, ultraviolet light.

Alternatively, the inorganic substrate may be first contacted with a fragrance composition, ink composition, or a mixture thereof, and thereafter subjected to electromagnetic radiation as described herein.

The following examples are presented to further illustrate the present invention and are not to be construed as unduly limiting the scope of this invention.

### EXAMPLE I

Examples I and II illustrate characteristics of fragrance materials of the present invention. As shown in Tables I-A and I-B below, 4 ounces of commercially available fragrance oil (Bergamot Fragrance Oil—Product Code Scent-0110, Scent-0110B, Scent-0101S-, Lone Star Candle Supply, Inc., Keller, Tex.) was mixed with 1 tablespoon of ink (commercially available from Best One Ink, Product Code Number 1010875, Tokyo, Japan) in a standard 1 quart paint-type container. The fragrance oil and ink were mixed together using a commercial paint mixing machine for 10 minutes.

TABLE I-A
Fragrance material

| | Principal | |
|---|---|---|
| Component | Components | Characteristics |
| Fragrance | Benzyl Acetate | Clear, light yellow mobile liquid with a |
| Oil | | citrus scent. |

TABLE I-B
Ink

| Trade Name | Chemical Name | Percent by Weight |
|---|---|---|
| Resin | Rosin Modified Phenolic Resin | 11-17% |
| Vegetable Oil | Linseed Oil | 11-17% |
| Petroleum | Petroleum Hydrocarbon | 52-58% |
| Hydrocarbon | | |
| Pigment | Organic/Inorganic Pigment | 8-17% |
| Wax | Polyethylene | Less than 2% |

### EXAMPLE II

1 pound of salt crystals (each individual salt crystal approximately 25 mm or less in size and commercially available from Saltworks, Inc. under the designation “RIO—Atlantic Sea Salt”) were subjected to microwave radiation (300 watts) for 3 minutes, and thereafter immersed in the ink/fragrance mixture prepared in accordance with EXAMPLE I. The salt crystals were then exposed to compressed air for approximately 1 minute while rotating the substrate to remove the excess ink/fragrance material. Thereafter, the crystals were then allowed to dry at ambient temperature overnight. Each of the crystals exhibited a bright vivid color, a strong scent, were dry to the touch and exhibited no surface residue.

### EXAMPLE III

This example illustrates properties of inorganic substrates prepared in accordance with the present invention. As shown in Table II below, approximately 1 pound of salt crystals (approximately 25 mm or less in size) (commercially available from Saltworks, Inc. under the trade designation “RIO”) were heated in a conventional oven at 350° F. for 10 minutes, and thereafter allowed to cool. These crystals referenced in Table II as CONTROL A, after being heated, appeared a dingy yellow color. The same amount and type crystals as used in CONTROL A (denoted in Table II as “B”) were subjected to microwave energy by placing the samples in a conventional microwave for approximately 3 minutes, and at 300 watts and allowed to cool. These crystals remained in the same state as provided by the manufacturer.

TABLE II

| | | Physical Characteristics/Color |
|---|---|---|
| RUN | Energy employed | after energized |
| Control | Thermal energy (conventional | Dingy yellow |
| A | oven, 350° F.) | |
| B | Microwave | White |

The results shown in the above examples, clearly demonstrate that the present invention is well adapted to carry out the objects and attain the ends and advantages mentioned as well as those inherent therein. Reasonable variations, modifications and adaptations may be made within the scope of this disclosure and the appended claims without departing from the scope of the invention.

What is claimed is:

1. A process for producing a fragrance material comprising:
pre-treating an inorganic substrate with electromagnetic radiation to form a pre-treated inorganic substrate, and
contacting the pre-treated inorganic substrate with at least one of a fragrance composition, an ink, and a mixture of a fragrance composition and an ink.

2. The process of claim 1, wherein the pre-treating step comprises passing the inorganic substrate through the path of the electromagnetic radiation.

3. The process of claim 1, wherein the pre-treating step comprises securing the inorganic substrate to a stationary point and passing electromagnetic radiation over or around the inorganic substrate.

4. The process of claim 1, wherein the contacting step comprises submerging the pre-treated inorganic substrate into a liquid mixture of fragrance material and ink.

5. The process of claim 1, wherein the fragrance composition and/or ink is dispersed throughout the inorganic substrate.

6. The process of claim 1, wherein the inorganic substrate contains sodium chloride.

7. The process of claim 1, wherein the inorganic substrate contains in the range of from about 90 to about 99 weight percent sodium chloride, based on the weight of the inorganic substrate.

8. The process of claim 1, wherein the electromagnetic radiation is selected from the group consisting of ultraviolet light, x-ray, infrared, and microwave radiation.

9. The process of claim 1, wherein the electromagnetic radiation is microwave radiation.

10. The process of claim 1, wherein the electromagnetic radiation has a frequency in the range of from about 1 gH to about 320 gH.

11. The process of claim 1, wherein the electromagnetic radiation has a frequency in the range of from about 1.5 gH to about 100 gH.

12. The process of claim 1, wherein the electromagnetic radiation has a frequency in the range of from about 2.0 gH to about 50 gH.

13. The process of claim 1, wherein the fragrance composition is present in an amount in the range of from about 1 to about 8 weight percent, based on the weight of the inorganic substrate.

14. The process of claim 1, wherein the ink is present in an amount in the range of from about 1 to about 8 weight percent, based on the weight of the inorganic substrate.

15. The process of claim 1, wherein the fragrance composition and/or ink is infused in the inorganic substrate.

16. The process of claim 1, wherein the fragrance material contains benzyl acetate.

17. The process of claim 1, wherein the ink contains a reducing agent in an amount in the range of from about 50 to about 60 weight percent, based on the weight of the ink.

18. A process for producing a fragrance material comprising:
contacting an inorganic substrate with a mixture containing a fragrance composition and an ink, and
subjecting the inorganic substrate to electromagnetic radiation sufficient to effect a cure thereof.

19. A fragrance material produced by the process of claim 1.

20. A fragrance material produced by the process of claim 18.

21. A fragrance material comprising a solid, semi porous inorganic substrate containing sodium chloride, wherein the inorganic substrate has been modified by electromagnetic radiation, a fragrance composition dispersed throughout the inorganic substrate, and an ink dispersed throughout the inorganic substrate.

22. The fragrance material of claim 1, wherein the fragrance material is used in a decorative manner.

23. The fragrance material of claim 1, wherein the fragrance material masks a pervading odor.
